(19) United States
(12) Patent Application Publication
(10) Pub. No.: US 2025/0279213 A1
(43) Pub. Date: Sep. 4, 2025

(54) IDENTIFICATION OF HEALTH RISK AND IMPACT ASSESSMENT

(71) Applicant: CITYBLOCK HEALTH, INC., Brooklyn, NY (US)

(72) Inventors: Azalea KIM, Brooklyn, NY (US); Emily LINDEMER, Reading, MA (US); Dhakshila PARAMANATHAN, Brooklyn, NY (US); Sarah DALTON, Seattle, WA (US); Myung WOO, Durham, NC (US); Michael TANG, Waban, MA (US); Eric HILTON, Whitefish, MT (US)

(21) Appl. No.: 19/067,272

(22) Filed: Feb. 28, 2025

Related U.S. Application Data
(60) Provisional application No. 63/560,309, filed on Mar. 1, 2024.

(51) Int. Cl. G16H 50/70; G16H 10/60; G16H 50/20; G16H 50/30

(52) U.S. Cl. CPC G16H 50/70; G16H 10/60; G16H 50/20; G16H 50/30

(57) ABSTRACT

Various examples of techniques for assessing patient risk and patient impact for health interventions are disclosed. In one example, a computer implemented method is disclosed that includes obtaining from a patient database patient information for a plurality of patients, the patient information including at least a risk score, clustering by a processor the plurality of patients based on the patient information using a categorization model, where the categorization model is trained using unsupervised learning, and assigning by the processor a risk category to each patient based on the clustering.

### CROSS-REFERENCE TO RELATED APPLICATIONS

This application claims the benefit of priority pursuant to 35 U.S.C. § 119 (e) to U.S. Provisional Patent Application No. 63/560,309, filed Mar. 1, 2024, entitled “Identification of Health Risk and Impact Assessment,” which is hereby incorporated by reference herein in its entirety. This application is related to U.S. application Ser. No. ______ [[Attorney Docket No. P316213.US.02]], filed on Feb. 28, 2025, entitled “Transition of Care Work Flow and Prioritization System,” which is hereby incorporated by reference herein in its entirety for all purposes.

### FIELD

The present disclosure relates generally to systems for categorizing patients based on health risks.

### BACKGROUND

Community health programs and patient outreach can significantly improve patient health outcomes. Patients with higher health needs benefit from more intensive care management through such programs. Identifying patients with higher health needs can be challenging. Various methods of identifying such patients are often subject to biases, which may result in under identification of some higher health needs patients, along with poorer health outcomes for such patients.

### SUMMARY

In one example, a computer implemented method is disclosed. The method includes obtaining from a patient database patient information for a plurality of patients, the patient information including at least a risk score, clustering by a processor the plurality of patients based on the patient information using a categorization model, where the categorization model is trained using unsupervised learning, and assigning by the processor a risk category to each patient based on the clustering.

In another embodiment, a method is disclosed that includes obtaining patient information for a plurality of patients, the patient information including at least a risk score and a diagnosis for each patient, clustering the plurality of patients based on the patient information using a categorization model, where the categorization model is trained using unsupervised learning, assigning a risk category to each patient based on the clustering, identifying of reach patient an impact of at least one intervention based on the diagnosis and using an impact model, and prioritizing at least a patient of the plurality of patients based on the risk category and the impact.

In yet another embodiment, a non-transitory computer readable media encoded with instructions, which when executed by one or more processors of a segmentation system is disclosed. The instructions cause the segmentation system to obtain patient information for a plurality of patients, the patient information including at least a risk score, cluster the plurality of patients based on the patient information using a categorization model, where the model is trained using unsupervised learning, and assigning a risk category to each patient based on the clustering.

In another embodiment, a computer implemented method is disclosed. The method includes obtaining patient information for a plurality of patients, the patient information including at least a diagnosis, determining using an impact model and based on the diagnosis an impact of at least one intervention based on the diagnosis, and prioritizing at least a patient of the plurality of patients based on the impact of the at least one intervention.

In another embodiment, a computer implemented method is disclosed. The method includes receiving a request for a patient profile, determining a risk category for the patient based at least on a risk score, where the risk category is determined using an unsupervised leaning model, and presenting via a user interface the patient profile where the patient profile includes a risk category for the patient.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. A more extensive presentation of features, details, utilities, and advantages of the present invention as defined in the claims is provided in the following written description of various embodiments and implementations and illustrated in the accompanying drawings.

### BRIEF DESCRIPTION OF THE DRAWINGS

FIG. 1 illustrates an example segmentation system and various systems in communication with the segmentation system through a network, in accordance with various embodiments of the disclosure.

FIG. 2 illustrates an example segmentation system in accordance with various embodiments of the disclosure.

FIG. 3 illustrates an example user interface of the segmentation system.

FIG. 4 illustrates an additional example user interface of the segmentation system.

FIG. 5 is a schematic diagram of an example computer system implementing various embodiments in the examples described herein.

FIG. 6 illustrates an example process for assigning risk categories to patients using the segmentation system.

FIG. 7 illustrates an example process for assessing impact of an intervention for a patient using the segmentation system.

FIG. 8 illustrates an example process for assigning risk category to a patient and assessing impact of an intervention for the patient using the segmentation system.

### DETAILED DESCRIPTION

Preventative healthcare, such as targeted outreach efforts, specialized programs, and the like generally decrease healthcare costs while improving health outcomes of patients. For example, patients participating in a program targeting management of a chronic condition may have fewer health complications related to the condition and may accrue less costs to treat the condition. However, identifying patients who are likely to most benefit from such efforts can be difficult. For example, identification of patients based on diagnosis codes (e.g., as utilized by insurance companies) may result in the inclusion of patients with well controlled chronic health conditions as high risk when such patients are less likely to benefit from outreach efforts. Identification of patients based on past healthcare utilization (e.g., by healthcare costs incurred) may result in the exclusion of patients with uncontrolled or poorly controlled chronic health conditions who have not historically utilized healthcare services, leading to poorer outcomes for those patients who may be excluded from targeted outreach efforts.

Further, existing models and systems of patient risk assessment may be inherently biased (e.g., biased based on race, gender, or other factors). Machine learning (ML) models may generally be less biased than manual or human implemented risk assessment. However, ML models may retain biases, which may decrease accuracy of the models. Some bias mitigation methods may identify underrepresented groups and may adjust outputs for such groups in some manner intended to mitigate potential bias. However, such methods are ineffective where data regarding membership in an underrepresented group is unavailable. Further, some types of data, such as healthcare utilization and cost of care, may be inherently biased against certain groups of individuals due to structural or societal inequities. Accordingly, models trained with and utilizing such data to categorize patients are inherently biased. Biased models are generally less accurate, such that the patients who would most benefit from targeted efforts may not be properly identified, resulting in poorer health outcomes and higher cost of care when compared to unbiased models.

The systems and methods described herein improve identification of patients who have a high likelihood of utilization of healthcare services and/or who may benefit from outreach efforts (e.g., by improving health outcomes, lowering healthcare costs, and the like). Models used herein may utilize multiple types of risk scores as input, generally reducing bias which would likely be present with manual sorting or utilizing models with different types of input. Healthcare utilization metrics used herein may also include inpatient events identified by both claims and admission-discharge-transfer (ADT) data (e.g., including data related to one or more admission, discharge, and/or transfer events) including overlapping events (e.g. one claim/health information exchange (HIE) event starts before another claim/HIE event has ended) for more accuracy. Elimination of bias generally more accurately categorizes patients, reducing overall healthcare costs for groups of patients and improving health outcomes through targeted outreach, preventative care, and/or other interventions.

Categorization of patients as described herein may result in cost savings and improve health outcomes for community health organizations and other organizations managing medical care for large groups of patients. For example, such organizations may tailor outreach and management based on the risk level of patients. The correct categorization of low risk patients may result in cost savings, as those patients generally do not need the higher, more costly levels of care. The correct categorization of high risk patients may result in improved health outcomes, as those patients are provided with more intensive care management and outreach.

The systems and methods described herein further identify patients who are particularly likely to benefit from inclusion in targeted programs or from increased outreach. For example, specific programs or care protocols may be used to manage behavioral health conditions which are not well controlled. The methods described herein may identify patients diagnosed with behavioral conditions who are most likely to see improvement from inclusion in such programs. Such identification may both improve medical outcomes for the patient population as a whole (e.g., by including patients who are likely to benefit from such programs) and control costs associated with such programs (e.g., by excluding patients who are less likely to benefit from such programs). The targeted outreach, follow-up, and/or other services identified may be assigned (e.g., dispatched) based on the results of the segmentation so as to treat those patients that are the highest risk and may be the most likely to benefit from the services (or other categories/weightings used by the segmentation system). It should be noted that various embodiments may be used to provide a general risk calculation that may be displayed on a user interface, e.g., top ten patient identifiers may be displayed to a nurse or may be stored within a patient database used for automated services follow-up.

Various embodiments of the present disclosure will be explained below in detail with reference to the accompanying drawings. Other embodiments may be utilized, and structural, logical and electrical changes may be made without departing from the scope of the present disclosure.

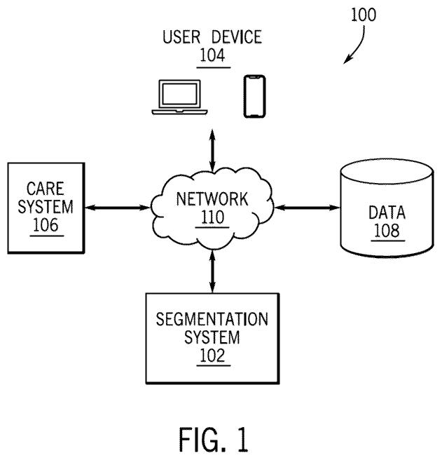

Turning now to the figures, FIG. 1 illustrates an example segmentation system 102 and various systems in communication with the segmentation system 102 through a network 110 As shown in FIG. 1, the segmentation system 102 may be accessed through a network 110 by various user devices 104. The segmentation system 102 may also communicate with various care systems 106 and data storage 108 through the network 110. For example, the segmentation system 102 may access various forms of patient data, information, risk scores, and the like from various care systems 106 and/or data storage 108 locations. The segmentation system may also communication with these same systems 106 and/or data storage locations 108, such as to output segmentation information for various patient populations to health services databases, provider databases, or the like.

The segmentation system 102 may generally utilize patient information to identify subgroups of patients or individual patients most likely to benefit from targeted intervention, outreach, and other preventative healthcare interventions. Such identification may include grouping patients based on health risks, such as likelihood of utilization of healthcare. In such examples, membership in a higher risk group may indicate that an individual or patient may benefit from additional resources and outreach. Further, the segmentation system 102 may assess which patients are most likely to be positively impacted by various outreach efforts or interventions.

In some examples, output from the segmentation system 102 may be utilized to prioritize patients or individuals for outreach or other healthcare interventions. For example, patients with a high risk of utilization and a high likelihood of positive impact of healthcare interventions may be contacted first for such interventions. In some examples, risk groups may be utilized to identify a course of action for an individual patient. For example, a patient in a low risk group may be contacted quarterly to discuss healthcare goals and may be scheduled for a yearly appointment with a primary care provider. A patient in a high risk group may be contacted more frequently, may be scheduled with various providers including specialists, and may be enrolled in targeted programs related to particular healthcare conditions. The cadence of the contact may be determined and output by the system to a user who can complete the actual outreach or may be automated, e.g., texts, calls, or the like.

The segmentation system 102 may be generally implemented by a computing device or combinations of computing resources in various embodiments. In various examples, the segmentation system 102 may be implemented by one or more servers, cloud computing resources, and/or other computing devices. The segmentation system 102 may, for example, utilize various processing resources to categorize and/or prioritize patients based on various patient data, e.g., updating a ranking of services for a patient database based on the segmentation system, flagging a patient in a patient database or services database for different or earlier services, or the like. The segmentation system 102 may further include memory and/or storage locations to store program instructions for execution by the processor and various data utilized by the segmentation system 102. The segmentation system 102 may be used to generate “call” or follow-up lists of patients based on provider or health services grouping to allow certain patients to be contacted by health providers earlier than others. The output lists may be stored in a database, transmitted via one or more notifications to various devices, and/or provided via a user interface.

User devices 104 may be utilized to provide input to, and receive output from, the segmentation system 102. For example, a user device 104 may belong to an end user tasked with patient outreach efforts, such as a health services provider. The user device 104 may be utilized to request categorization of a particular patient (e.g., placement of the patient into a particular segment), prioritization or categorization of a group of patients, and the like. The user device 104 may receive categorizations, identifications of particular programs or outreach efforts for a patient, and other output from the segmentation system 102.

Generally, the user device 104 and/or other user devices in communication with the segmentation system 102 may be devices belonging to an end user utilizing the segmentation system 102, such as end users tasked with patient outreach efforts and the like. In some implementations, many user devices 104 may access the segmentation system 102 to view a segment or grouping for a patient, prioritize patients in a group of patients, view interventions likely to have a positive impact on a patient, and perform other functions. In various embodiments, user devices 104 may be authenticated by an authentication service prior to accessing the segmentation system 102.

In various implementations, the user device 104 and/or other user devices in communication with the segmentation system 102 may be implemented using any number of computing devices including, but not limited to, a computer, a laptop, mobile phone, smart phone, wearable device (e.g., AR/VR headset, smart watch, smart glasses, or the like), smart speaker, vehicle (e.g., automobile), or appliance. Generally, the user devices may include one or more processors, such as a central processing unit (CPU) and/or graphics processing unit (GPU). The user devices may generally perform operations by executing executable instructions (e.g., software) using the processor(s).

A care system 106 may be in communication with the segmentation system 102. In various examples, multiple care systems 106 may be in communication with the segmentation system 102 via the network 110. Various care systems 106 in communication with the segmentation system 102 may be associated with or utilized by insurance companies, healthcare providers, healthcare facilities, or other healthcare related entities. For example, care systems 106 may include electronic health record (EHR) systems, insurance claim systems, and other systems storing or utilizing patient data. In some examples, care systems 106 may further be systems associated with entities providing interventions or administering programs recommended by the segmentation system 102. Such care systems 106 may utilize output data from the segmentation system 102.

Various data utilized by the segmentation system 102 may be stored at data storage 108. For example, various types of patient information or patient data may be stored at data storage 108. Patient profiles, including patient age, diagnoses, categorizations, and/or additional patient data may be stored at data storage 108. The segmentation system 102 may both retrieve data from and write data to data storage 108.

The network 110 may be implemented using one or more of various systems and protocols for communications between computing devices. In various embodiments, the network 110 or various portion of the network 110 may be implemented using the Internet, a local area network (LAN), a wide area network (WAN), and/or other networks. In addition to traditional data networking protocols, in some embodiments, data may be communicated according to protocols and/or standards including near field communication (NFC), Bluetooth, cellular connections, and the like.

Components of the segmentation system 102 and those in communication with the segmentation system 102 shown in FIG. 1 are exemplary and may vary in some embodiments. For example, in some embodiments, the segmentation system 102 may be distributed across multiple computing elements, such that components of the segmentation system 102 communicate with one another through the network 110. Further, in some embodiments, computing resources dedicated to the segmentation system 102 may vary over time based on various factors such as usage of the segmentation system 102. In some embodiments, the segmentation system 102 may communicate with multiple user devices 104 and/or other systems not shown in FIG. 1.

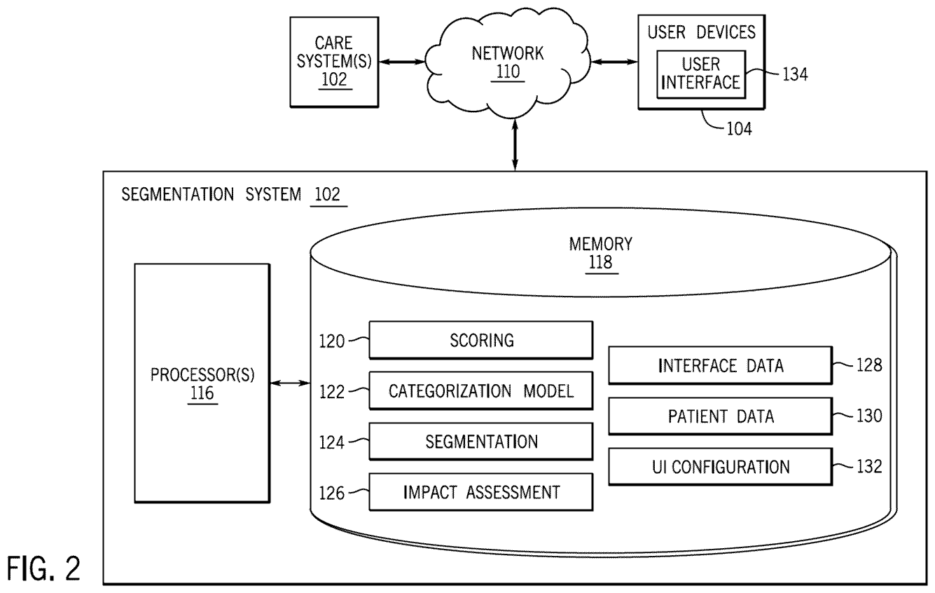

FIG. 2 illustrates an example segmentation system 102. The segmentation system 102 generally stratifies, triages, or sorts patients according to predicted intensity of medical care and types of needs. Such stratification may help to prioritize preventative care and outreach to patients with higher medical or behavioral health needs. For example, the segmentation system 102 may categorize a patient or group of patients based on likelihood of healthcare utilization (e.g., low likelihood, rising likelihood, and high likelihood) based on risk scores and other patient information.

In various examples, the segmentation system 102 may include or utilize one or more hosts or combinations of compute resources, which may be located, for example, at one or more servers, cloud computing platforms, computing clusters, and the like. Generally, the segmentation system 102 is implemented by compute resources including hardware for memory 118 and one or more processors 116. For example, the segmentation system 102 may utilize or include one or more processors, such as a CPU, GPU, and/or programmable or configurable logic.

In some embodiments, various components of the segmentation system 102 may be distributed across various computing resources, such that components of the segmentation system 102 communicate with one another through the network 110 and/or using other communications protocols. For example, in some embodiments, the segmentation system 102 may be implemented as a serverless service, where computing resources for various components of the segmentation system 102 may be located across various computing environments (e.g., cloud platforms) and may be reallocated dynamically and/or automatically according to, for example, resource usage of the segmentation system 102. In various implementations, the segmentation system 102 may be implemented using organizational processing constructs such as functions implemented by worker elements allocated with compute resources, containers, virtual machines, and the like.

In various examples, the memory 118 may directly store various types of data and/or instructions for accessing such data from another location or source. For example, memory 118 may store patient data 130. Patient data 130 may generally be used to categorize patients, identify patients for inclusion in particular programs, generate user interfaces to the segmentation system 102, and the like. In various examples, patient data may include biographical data (e.g., name, age, contact information, sex, and the like), treatment data (e.g., information about healthcare appointments, diagnoses, providers, and the like), intervention data (e.g., contacts with the patients, interventions already given to the patient, and the like), insurance data, risk scores, assigned segment, and the like. Such patient data 130 may be utilized by the segmentation system 102 to categorize or assign patients to segments, prioritize patients for particular interventions, present profiles or user interfaces to the segmentation system 102, and to perform other functions of the segmentation system 102. In addition to be displayed on various user interfaces, such as to health services providers that may use such information to determine action items for their day, the data may also be stored in one or more databases that are utilized to help manage health services for patients. For example, the segmentation risk may be used to select patient lists for follow-up with health providers, such as calls or visits.

Memory 118 may further store interface data 128. Interface data 128 may include instructions, locations, or other information to retrieve data from or provide data to various systems in communication with the segmentation system 102, such as care systems 106. For example, interface data 128 may include types of data stored at a care system 106, a location of the care system 106, and specific types of calls or commands that may be used to access the data stored at the care system 106 or other computing system with an interface to the segmentation system 102.

The memory 118 may further include (e.g., store or access) instructions for various functions of the segmentation system 102 which, when executed by processor 116, perform various functions of the segmentation system 102. The memory 118 may further store data and/or instructions for retrieving data used by the segmentation system 102. Similar to the processor 116, memory resources utilized by the segmentation system 102 may be distributed across various physical computing devices. In some examples, memory 118 may access instructions and/or data from other devices or locations, and such instructions and/or data may be read into memory 118 to implement the segmentation system 102.

In various embodiments, memory 118 may store instructions for scoring 120. When executed by the processor 116, the instructions for scoring 120 generally cause the processor to determine or retrieve (e.g., from patient data 130) one or more risk scores for a patient or group of patients. Such risk scores may be utilized as input to the categorization model 122 and may be chosen to reduce bias in the categorization model 122. Risk scores may include, in various examples, hierarchical condition category (HCC) risk scores, chronic illness and disability payment system (CDPS) risk scores, Elixhauser mortality scores, Elixhauser readmission scores, inpatient (IP) and emergency department (ED) likelihood scores, and patient age.

In some embodiments the IP and ED likelihood scores may come from a database with external data. Alternatively or additionally, the IP and ED likelihood scores may come from a machine learning model, such as one trained on historical data related to IP and ED visits and readmissions or similar events. For example, the IP and ED likelihood score models may predict six-month likelihoods of inpatient admission and of emergency department visits the patient or group of patients. These models may utilize reconciled data across claims, ADT data, and HIE/EHR sources to predict a comprehensive recent medical history for the patient or group of patients. The models' features, or input variables, include detailed information about recent acute and outpatient utilization, about diagnoses captured and procedures conducted, demographics, and the like. The models may be random forest classifiers that use sample weighting to address class imbalance (e.g., low percentage of positive cases). This helps to ensure that the trained models are not biased to a certain balance of readmissions or other outcomes that may be prevalent due to the skewed training data, e.g., more data may be available for patients who are readmitted as compared to those who are not just due to data collection methods. Similarly, the models may use grid search to optimize hyperparameters. The models may, for example, be trained on 80% of the observations (e.g., index encounters) and performance may be measured on the remaining 20% (e.g., the test set). This again, helps to prevent inadvertent skewing the predictive abilities of the trained models.

The risk scores utilized may reduce bias in the categorization model 122. The risk scores are generally industry standard risk scores which are validated for healthcare use, are not defined only by past utilization, and are agnostic of healthcare costs. Scores that are defined only by past use or utilization may increase bias as differences in access and trust of the healthcare system impact how different groups interact with the healthcare system. Scores that utilize healthcare costs may increase bias by underestimating need in protected groups. Accordingly, use of industry standard scores which are not defined only by past utilization and which are agnostic of healthcare costs generally reduces bias in the output of the categorization model 122.

Memory 118 may further store a categorization model 122. The categorization model 122 may be an unsupervised clustering algorithm (e.g., k means clustering or other unsupervised algorithms) configured to output clusters of patients based on the patient information provided as input. For example, the categorization model 122 may generate four clusters thought to represent low risk for utilization for physical health, low risk for utilization for behavioral health, high risk for utilization for physical health, and high risk for utilization for behavioral health. In various examples, the number of clusters may vary and may be provided to the categorization model 122 as a changeable parameter.

The categorization model 122 may utilize various types of patient information as input, generally including at least one risk score for the patient. Such patient information may include, in various examples, patient age, history of hospital admissions, diagnoses, medical history, and the like. Generally, the categorization model also utilizes one or more risk scores generated by scoring 120 and/or stored as patient data 130. In one example, the categorization model 122 receives, for each patient, a HCC score, CDPS score, Elixhauser mortality score, Elixhauser readmission score, inpatient and ED likelihood scores (either as data received from a database or based on trained ML models). The categorization model 122 may then use a clustering algorithm (e.g., k means clustering) to cluster members into some number of clusters, where each cluster approximates some level of risk for healthcare utilization.

Instructions for segmentation 124 may further be stored at memory 118. When executed by the processor 116, the instructions for segmentation generally utilize output of the categorization model and, in some examples, additional rules or guidelines to assign patients to segments (e.g., risk categories). For example, segmentation may receive clusters or cluster assignments for members from the categorization model 122. Segmentation 124 may then move individual patients or groups of patients to other clusters, create new groupings, combine clusters into a single grouping, or the like.

In one particular example, the categorization model 122 may group patients into four groupings including high risk associated with behavioral health, high risk associated with physical health, low risk associated with behavioral health, and low risk associated with physical health. Segmentation 124 may be configured to group patients into segments representing high risk associated with behavioral health, high risk associated with physical health, rising risk associated with behavioral health, rising risk associated with physical health, and low overall risk. Segmentation 124 may group patients into segments based on a variety of factors. For example, segmentation 124 may move patients with particular diagnoses from the low risk associated with behavioral health to rising risk associated with behavioral health. Segmentation 124 may move patients from the low risk associated with behavioral health to rising risk associated with behavioral health based on additional factors, such as percentile likelihood of emergency department or inpatient admission. For example, patients above the 75th percentile for likelihood of inpatient admission or patients above the 75th percentile for likelihood of admission to the emergency department may be moved to rising risk associated with behavioral health. Similarly, segmentation 124 may move patients from low risk associated with physical health to rising risk associated with physical health based on patient percentile. For example, patients above the 75th percentile for likelihood of inpatient admission or patients above the 75th percentile for likelihood of admission to the emergency department may be moved to rising risk associated with physical health. Segmentation 124 may further combine low risk associated with behavioral health and low risk associated with physical health into a single low risk category.

Memory 118 may further store instructions for impact assessment 126. When executed by the processor 116, instructions for impact assessment may cause the processor to evaluate patients for particular interventions and prioritize patients for particular interventions. In various examples, impact assessment 126 may utilize an impact model to determine an impact of a particular intervention for a patient based on various patient data 130 associated with the patient. For example, an impact model may be a machine learning model configured to output an impact score or other indication of potential impact based on an input of a diagnosis of a patient. In some examples, impact assessment 126 may be used to assess potential impact of an intervention for a single patient at a time. In some examples, impact assessment 126 may further prioritize multiple patients. For example, impact assessment 126 may obtain impact scores for multiple patients and may rank the multiple patients, with those more likely to be positively impacted by an intervention ranked higher. In some examples, the impact model may utilize unsupervised learning techniques (e.g., clustering) to prioritize patients in a group of patients. In some examples, the impact model may be trained using supervised learning techniques to generate impact scores based on patient data 130.

Instructions for user interface configuration 132 may be stored at the memory 118. When executed by the processor 116, the instructions for user interface configuration 132 cause the processor 116 to configure various user interfaces to display information generated by the segmentation system 102 and to transmit such user interfaces for display at user interfaces 134 of various user devices 104. For example, user interface configuration 132 may receive a patient category or segment from segmentation 124 and may configure a user interface displaying the category or segmentation for a patient or group of patients. User interface configuration 132 may similarly receive various information from patient data 130 in order to configure various user interfaces for presentation at user interfaces 134 of user devices 104 in communication with the segmentation system 102.

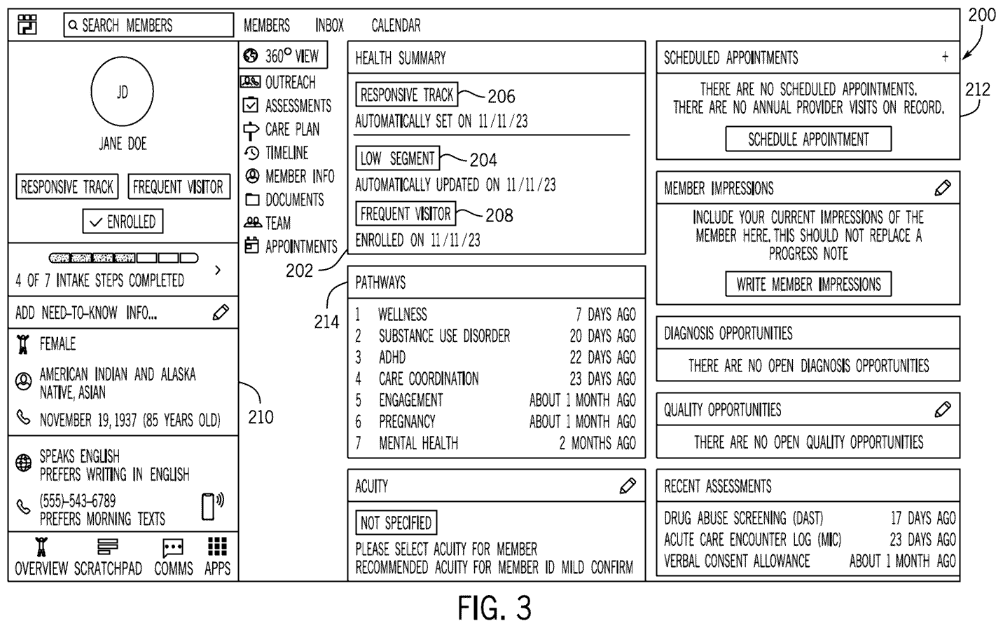

FIG. 3 illustrates an example user interface 200 of the segmentation system 102. The user interface 200 may generally be fully or partially configured by user interface configuration 132 and may be presented at a user interface 134 of a user device 104 in communication with the segmentation system 102. The user interface 200 generally shows a user profile of a patient categorized or assigned a segment using the segmentation system 102. For example, the user interface 200 may display a health summary 202 including an assigned segment or category 204. The segment 204 shown in the user interface shows that the patient was assigned to a low risk category by the segmentation system 102. The health summary 202 further shows that the patient has been placed on a responsive track 206, which may be a defined track or plan of care for patients in the low risk segment or category.

In various examples, elements such as the segment 204, track 206, and utilization status 208 may be colored or patterned according to a patient's category. For example, in the user interface 200, the track 206 may be green to illustrate that the track is associated with low risk patients and requires less attention from the user. Similarly, the segment 204 may be colored green or otherwise patterned to indicate that the patient is in the low risk segment. Other segments may be colored or patterned differently. For example, a rising risk segment may be colored orange or yellow, while a high risk segment may be colored red. As shown in the user interface 200, the patient's utilization status may similarly be colored or patterned based on the patient's level of healthcare utilization.

The user interface 200 may include additional elements in various examples. Such additional elements may be populated using various patient data 130 stored at the segmentation system 102 and/or accessible by the segmentation system 102 and stored at other data storage 108. For example, biographical data 210, appointment data 212, and treatment pathways 214 are displayed at the user interface 200. In some examples, the user interface 200 may further include selectable elements to send requests or commands to the segmentation system 102. For example, via the user interface 200, a user may request that a segment or categorization for a user be recalculated or updated using the segmentation system 102. A user may further, through the user interface 200, update various patient information and such patient information may be provided to the segmentation system 102 to be stored as patient data 130.

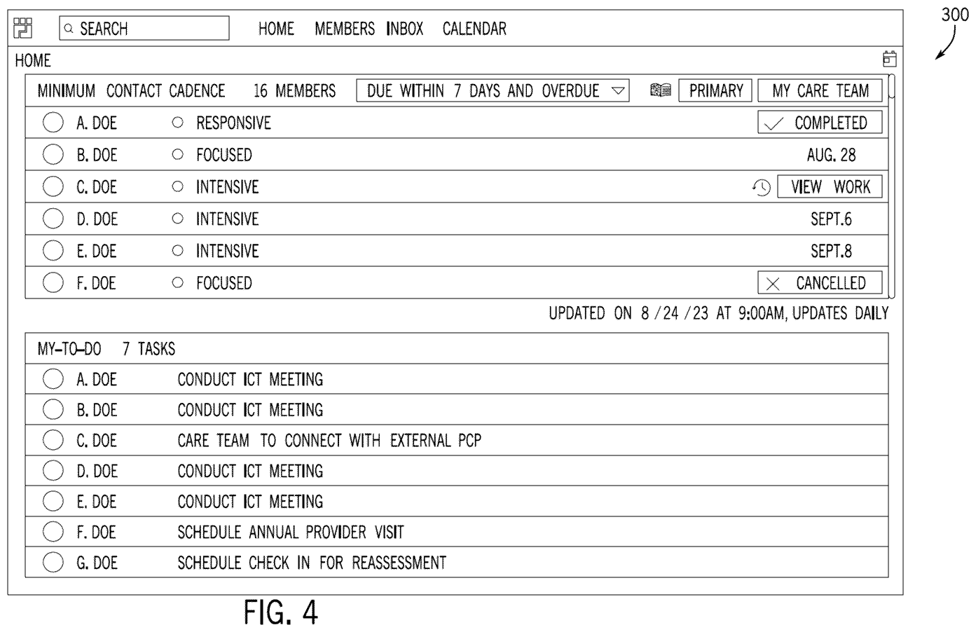

FIG. 4 illustrates an additional example user interface 300 of the segmentation system 102. The user interface 300 may generally be fully or partially configured by user interface configuration 132 and may be presented at a user interface 134 of a user device 104 in communication with the segmentation system 102. For example, the user interface 300 may be utilized by a user responsible for outreach efforts. Such users may utilize information generated by the segmentation system 102 to, for example, develop care plans based on segments generated by the segmentation system 102, identifying patients who may be good candidates for particular interventions based on impact scores or other measures of impact generated by the segmentation system 102, and the like. For example, the user interface 300 may display a list of tasks including patients to contact or specific tasks to perform for patients. Such tasks may be based on segmentation or other information generated by the segmentation system 102. For example, the frequency of contact for a patient may be set based on the assigned segment of the patient. Similarly, the tasks to be performed for each patient may be selected based on either or both of the segmentation or identified interventions generated by the segmentation system 102.

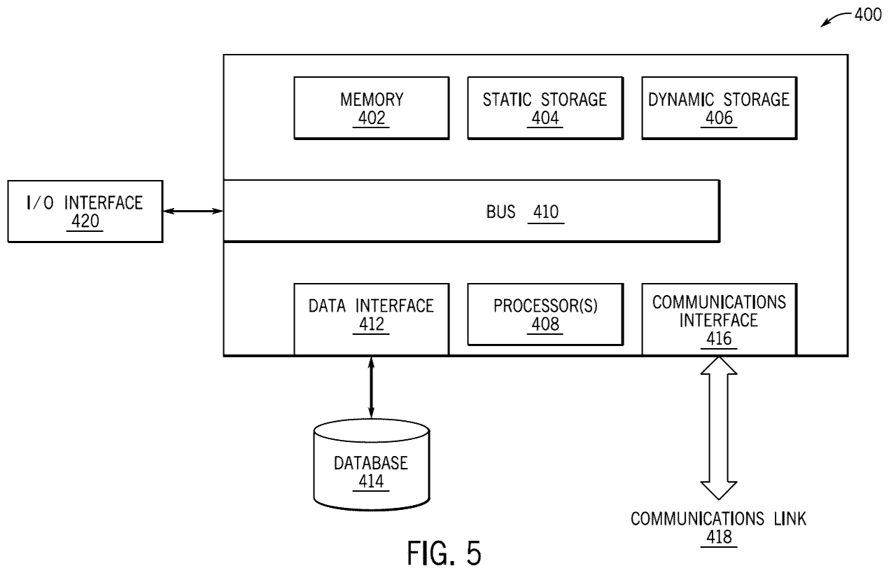

The segmentation system 102 may be implemented using various computing systems. Turning to FIG. 5, an example computing system 400 may be used for implementing various embodiments in the examples described herein. For example, processor 116 and memory 118 may be located at one or several computing systems 400. In various embodiments, user device 104 is also implemented by a computing system 400. This disclosure contemplates any suitable number of computing systems 400. For example, the computing system 400 may be a server, a desktop computing system, a mainframe, a mesh of computing systems, a laptop or notebook computing system, a tablet computing system, an embedded computer system, a system-on-chip, a single-board computing system, or a combination of two or more of these. Where appropriate, the computing system 400 may include one or more computing systems; be unitary or distributed; span multiple locations; span multiple machines; span multiple data centers; or reside in a cloud, which may include one or more cloud components in one or more networks.

Computing system 400 includes a bus 410 (e.g., an address bus and a data bus) or other communication mechanism for communicating information, which interconnects subsystems and devices, such as processor 408, memory 402 (e.g., RAM), static storage 404 (e.g., ROM), dynamic storage 406 (e.g., magnetic or optical), communications interface 416 (e.g., modem, Ethernet card, a network interface controller (NIC) or network adapter for communicating with an Ethernet or other wire-based network, a wireless NIC (WNIC) or wireless adapter for communicating with a wireless network, such as a WI-FI network), and an input/output (I/O) interface 420 (e.g., keyboard, keypad, mouse, microphone). In particular embodiments, the computing system 400 may include one or more of any such components.

In particular embodiments, processor 408 includes hardware for executing instructions, such as those making up a computer program. The processor 408 circuitry includes circuitry for performing various processing functions, such as executing specific software for performing specific calculations or tasks. In particular embodiments, I/O interface 420 includes hardware, software, or both providing one or more interfaces for communication between computing system 400 and one or more I/O devices. Computing system 400 may include one or more of these I/O devices, where appropriate. One or more of these devices may enable communication between a person and computing system 400.

In particular embodiments, communications interface 416 includes hardware, software, or both providing one or more interfaces for communication (such as, for example, packet-based communication) between computing system 400 and one or more other computer systems or one or more networks. One or more memory buses (which may each include an address bus and a data bus) may couple processor 408 to memory 402. Bus 410 may include one or more memory buses, as described below. In particular embodiments, one or more memory management units (MMUs) reside between processor 408 and memory 402 and facilitate access to memory 402 requested by processor 408. In particular embodiments, bus 410 includes hardware, software, or both coupling components of the computing system 400 to each other.

Accordingly to particular embodiments, computing system 400 performs specific operations by processor 408 executing one or more sequences of one or more instructions contained in memory 402. For example, instructions for scoring 120, the categorization model 122, segmentation 124, and impact assessment 126 may be contained in memory 402 and may be executed by the processor 408. Such instructions may be read into memory 402 from another computer readable/usable medium, such as static storage 404 or dynamic storage 406. In alternative embodiments, hard-wired circuitry may be used in place of or in combination with software instructions. Thus, particular embodiments are not limited to any specific combination of hardware circuitry and/or software. In various embodiments, the term “logic” means any combination of software or hardware that is used to implement all or part of particular embodiments disclosed herein.

The term “computer readable medium” or “computer usable medium” as used herein refers to any medium that participates in providing instructions to processor 208 for execution. Such a medium may take many forms, including but not limited to nonvolatile media and volatile media. Non-volatile media includes, for example, optical or magnetic disks, such as static storage 404 or dynamic storage 406. Volatile media includes dynamic memory, such as memory 402.

Computing system 400 may transmit and receive messages, data, and instructions, including program, e.g., application code, through communications link 418 and communications interface 416. Received program code may be executed by processor 408 as it is received, and/or stored in static storage 404 or dynamic storage 406, or other storage for later execution. A database 414 may be used to store data accessible by the computing system 400 by way of data interface 412. For example, interface data 128 and patient data 130 may be stored using a database 414. In various examples, communications link 418 may communicate with, for example, user devices to display user interfaces to the segmentation system 102.

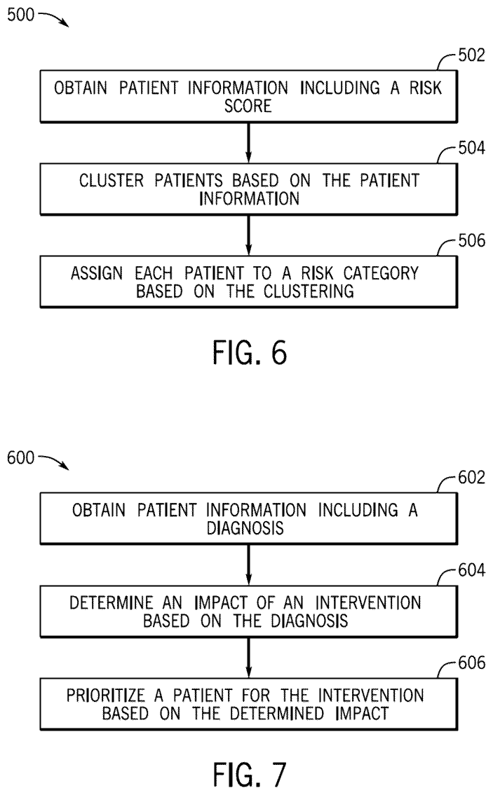

FIG. 6 illustrates an example process 500 for determining risk categories to patients using the segmentation system 102. At block 502, the segmentation system 102 obtains patient information including a risk score. In some examples, the patient information may be obtained from an external source. For example, patient information may be obtained from one or more care systems 106 and/or one or more data storage locations. Generally, the segmentation system 102 may utilize interface data 128 to obtain such information from various locations. For example, the segmentation system 102 may utilize interface data 128 to make a call to a care system 106 to obtain patient information. Further, some patient information may be stored at the segmentation system 102 as patient data. In some examples, one or more of the risk scores used by the segmentation system 102 may be obtained as patient information (e.g., from a care system 106). One or more of the risk scores used by the segmentation system 102 may further be calculated by the segmentation system 102 based on other patient information obtained at block 502. For example, scoring 120 may generate one or more risk scores for a patient or patients based on patient information. In some examples, the risk scores may be stored as patient data 130 for later use by the segmentation system 102.

In some examples, the segmentation system 102 may obtain patient information for one patient at block 502. For example, the segmentation system 102 may receive a request (e.g., through a user interface to the segmentation system 102) to determine a segment or categorization for a single patient. In other examples, the segmentation system 102 may obtain patient information for multiple patients. For example, the segmentation system 102 may update segments for an entire group of patients at various intervals (e.g., every month) and may obtain updated patient information for each patient in the group of patients when updating the segments.

The segmentation system 102 clusters patients based on the patient information at block 504. The segmentation system 102 generally uses the categorization model 122 to cluster patients based on the patient information. For example, patient information for each patient to be included in the clustering may be provided to the categorization model 122 as input. The categorization model 122 may use various types of clustering algorithms to cluster patients into any number of clusters. In one example, the categorization model 122 uses k-means clustering to generate four patient clusters, where the patient clusters generally represent patients having a low behavioral health risk, patients having a low physical health risk, patients having a high behavioral health risk, and patients having a high physical health risk. The number of clusters may generally be provided as input to the categorization model 122 and may be based on the desired number of segments or categories of patients or other factors.

At block 506, the segmentation system 102 assigns each patient to a risk category based on the clustering. Segmentation 124 may utilize the clustering from the categorization model 122 to assign patients to a risk category. In some examples, the risk categories may correlate directly with the clusters generated by the categorization model 122. That is, each cluster may correspond to a risk category or segment. In other examples, segmentation 124 may utilize the clusters as a starting point for segmentation into more or fewer segments or categories than the number of clusters. Segmentation 124 may apply various rules to categorize patients based on the clustering. For example, where the categorization model 122 groups patients into four categories roughly representing high behavioral health risk, high physical health risk, low behavioral health risk, and low physical health risk, segmentation 124 may combine the low behavioral health risk and low physical health risk clusters to obtain one low risk category. Segmentation 124 may further move patients from the low risk category to rising behavioral health risk or rising physical health risk based on various factors including, for example, specific diagnoses. Accordingly, segmentation 124 may generate a different number of segments or categories than the number of clusters generated by the categorization model 122.

In various examples, the segments or categories generated using the process 500 may be presented to a user (e.g., at a user device 104 via a user interface 134). For example, a patient profile may include a patient risk category (e.g., as shown in the user interface 200). In some examples, the segment or risk category may further be used to configure other aspects of a user interface. For example, the segmentation system 102 may output a recommended action based on an assigned risk category. For example, the user interface may output a recommended check in with the patient based on risk category, a notification, or other tag that may be used to generate notifications or action items within a health care platform or system.

In some examples, the segments or categories may be stored as patient data 130 and/or stored at a database 108. In such examples, users may be tagged with a risk category and the risk category may be displayed after a triggering event, such as hospital admission, a request to view a patient profile, a medical appointment, or the like. The segments may further be utilized by end users to, for example, establish a schedule for contact with the patient, a care plan for the patient, identify particular interventions for the patient, and the like.

FIG. 7 illustrates an example process 600 for assessing impact of an intervention for a patient using the segmentation system 102. At block 602, the segmentation system 102 obtains patient information including a diagnosis. Such patient information may be stored as patient data 130 and/or at a database 108 remote from the segmentation system 102. In some examples, patient information may further be obtained by querying one or more care systems 106. For example, a diagnosis may be obtained from a care system 106, such as an EHR associated with a healthcare provider while other information may be obtained from another care system 106 associated with another healthcare provider, an insurance company, or the like. In such examples, interface data 128 may be used to query various care systems 106 to obtain the relevant patient information.

The segmentation system 102 determines an impact of an intervention based on the diagnosis at block 604. In various examples, an intervention may be a program specifically targeted towards improving a particular diagnosis or condition. Accordingly, patients having the diagnosis or condition may be identified as candidates for the intervention or program. In some examples, additional screening and/or analysis may identify a subset of patients having a diagnosis who are more likely to benefit from a particular intervention.

The impact of the intervention may be based on the diagnosis and various other factors, such as the severity of the diagnosis, control of the condition, comorbidities, historical participation of the patient in various interventions, and the like. For example, a patient with well controlled diabetes may be less likely to experience health improvements from a program aimed at lifestyle interventions for control of diabetes. In another example, patients with significant comorbidities, including terminal diagnoses, may be less likely to experience health improvements from such programs. Further, patients who have already participated in similar interventions may be less likely to experience health improvements from repeating the same or a similar program. Such additional factors may be derived from various patient information, including patient information previously stored as patient data 130. In various examples, the impact of a diagnosis may be based on a numerical impact score, ranking of the patient, or the like. The impact may include a trained ML model that predicts the likelihood of a positive impact based on the various characteristics of the patient and the diagnosis.

At block 606, the segmentation system 102 prioritizes a patient for the intervention based on the determined impact. In various examples, prioritization may including ranking patients from those most likely to benefit from an intervention to those least likely to benefit from an intervention. Such rankings may be displayed, in various examples, via a user interface 134 of a user device 104 accessing the segmentation system 102. For example, an end user responsible for contacting patients for inclusion in a program or intervention may be provided with a listing of patients to contact and may first contact patients who are more likely to be positively impacted by participation in such an intervention. Accordingly, such programs are more likely to improve health outcomes for patients included in the programs. Further, such prioritization improves resource allocation, as including the most impacted patients is most likely to reduce healthcare spending on management of various medical conditions. In some embodiments, the patient ranking or some grouping of patients (e.g., the highest risk 25% of a patient population) may be transmitted to other databases, such as to other health care providers or the like.

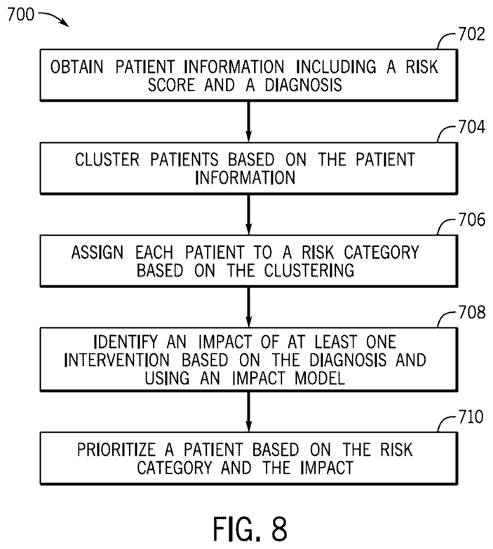

FIG. 8 illustrates and example process 700 for assigning a risk category to a patient as assessing an impact of an intervention for the patient using the segmentation system 102. At block 702, the segmentation system 102 obtains patient information including a risk score and a diagnosis. Such patient information may be obtained from stored patient data 130 and/or from external sources. For example, patient information may be stored at one or more databased 108 and/or may be obtained from a care system 106. For example, interface data 128 may be used by the segmentation system 102 to query a care system 106 or multiple care systems 106 to obtain patient information. In some examples, scoring 120 may further generate one or more risk scores at block 702, and such risk scores may be included in patient information. The risk scores may, in some examples, be stored as patient data 130.

The segmentation system 102 clusters patients based on the patient information at block 704. Generally, the categorization model 122 may cluster patients based on patient information. The segmentation system 102 may cluster the patients using the same or similar methods as those described with respect to block 504 of the method 500. For example, an unsupervised learning algorithm, such as k-means clustering, may be used to generate a number of patient clusters representing differing levels of patient risk.

At block 706, the segmentation system 102 assigns each patient to a risk category based on the clustering. Generally, segmentation 124 utilizes the clustering from the categorization model 122 to assign patients to a risk category. The segmentation system 124 may assign patients to risk categories using the same or similar methods as those described with respect to block 506 of the method 500. For example, segmentation 124 may combine clusters, create new categories by moving particular patients into new risk categories, and the like.

The segmentation system 102 identifies an impact of at least one intervention based on the diagnosis at block 708. In some examples, the segmentation system 102 may evaluate patients in one or several risk categories for a particular intervention without evaluating patients in other risk categories. Such evaluation by risk category may reduce time and processing resources used to evaluate impact of various interventions. For example, when evaluating impact of an intensive program for treatment of behavioral disorders, the segmentation system 102 may first evaluate those patients in the high risk category for behavioral health and may not evaluate patients in the low risk category. In some examples, the segmentation system 102 may first evaluate patients in the high risk category before evaluating patients in lower risk categories.

At block 710, the segmentation system 102 prioritizes a patient based on the risk category and the impact. The segmentation system 102 may prioritize patients based on impact using similar or the same methods described with respect to block 606 of the process 600. The segmentation system 102 may further prioritize based on risk category. For example, higher risk category patients may be prioritized over lower risk category patients, where the prioritization may be within a services lists, database, or the like. In some examples, prioritization may include ranking patients based on impact and/or risk category or segment. Such rankings may be displayed, in various examples, via a user interface 134 of a user device 104 accessing the segmentation system 102. For example, the user interface 300 may display patients on a “to-do list” based on the prioritization, e.g., a list of patients for immediate follow-up by a service provider (health worker). The rankings may also be stored in a services or health services database utilize to assign and schedule health services.

In various examples, the segmentation system 102 may interface with other software (e.g., care management software) that uses the risk categories generated by the segmentation system 102. For example, the segmentation system 102 may provide risk categories to systems utilized to manage patient care. In such examples, such systems may suggest specific interventions or actions based on a patient's risk category. Such systems may further generate alerts based on a patient's risk category or changes in the patient's risk category. For example, an alert may be triggered notifying a patient's primary healthcare provider when the segmentation system 102 categorizes the patient as high risk.

According to the above examples, the segmentation system 102 may help to ensure that outreach efforts, interventions, care management, and other health programs are carried out in a manner that increases health outcomes for patients while reducing cost of administering such programs. For example, high risk patients may be identified for more intensive care management, improving their health outcomes. Similarly, low risk patients may be identified and provided with less intensive care management, reducing the costs associated with providing healthcare to low risk patients. Currently such patient risk identification and services triaging is not possible given variation between different providers, patient characteristics, and extensive databases.

The technology described herein may be implemented as logical operations and/or modules in one or more systems. The logical operations may be implemented as a sequence of processor-implemented steps directed by software programs executing in one or more computer systems and as interconnected machine or circuit modules within one or more computer systems, or as a combination of both. Likewise, the descriptions of various component modules may be provided in terms of operations executed or effected by the modules. The resulting implementation is a matter of choice, dependent on the performance requirements of the underlying system implementing the described technology. Accordingly, the logical operations making up the embodiments of the technology described herein are referred to variously as operations, steps, objects, or modules. Further, it should be understood that logical operations may be performed in any order, unless explicitly claimed otherwise or a specific order is inherently necessitated by the claim language.

In some implementations, articles of manufacture are provided as computer program products that cause the instantiation of operations on a computer system to implement the procedural operations. One implementation of a computer program product provides a non-transitory computer program storage medium readable by a computer system and encoding a computer program. It should further be understood that the described technology may be employed in special purpose devices independent of a personal computer.

The above specification, examples, and data provide a complete description of the structure and use of exemplary embodiments of the invention as defined in the claims. Although various embodiments of the claimed invention have been described above with a certain degree of particularity, or with reference to one or more individual embodiments, it is appreciated that numerous alterations to the disclosed embodiments without departing from the spirit or scope of the claimed invention may be possible. Other embodiments are therefore contemplated. It is intended that all matter contained in the above description and shown in the accompanying drawings shall be interpreted as illustrative only of particular embodiments and not limiting. Changes in detail or structure may be made without departing from the basic elements of the invention as defined in the following claims.

1. A computer implemented method for providing health services comprising:
obtaining from a patient database patient information for a plurality of patients, the patient information including at least a risk score;
clustering by a processor the plurality of patients based on the patient information using a categorization model, wherein the categorization model is trained using unsupervised learning;
assigning by the processor a risk category to each patient based on the clustering; and
outputting to a computing device a recommended action based on the assigned risk category.

2. The method of claim 1, wherein obtaining the patient information comprises accessing the patient information at least one remote health care database and wherein the patient information includes a plurality of risk scores.

3. The method of claim 1, wherein the risk score is based on insurance claims data.

4. The method of claim 1, wherein the risk score is agnostic to healthcare costs.

5. The method of claim 2, further comprising calculating at least one risk score of the plurality of risk scores based on the patient information.

6. The method of claim 1, further comprising prioritizing the plurality of patients based on the risk category assigned to each patient.

7. A method for managing health services comprising:
obtaining patient information for a plurality of patients, the patient information including at least a risk score and a diagnosis for each patient;
clustering the plurality of patients based on the patient information using a categorization model, wherein the categorization model is trained using unsupervised learning;
assigning a risk category to each patient based on the clustering;
identifying, for each patient, an impact of at least one intervention based on a diagnosis and using an impact model; and
prioritizing in a health services database at least a patient of the plurality of patients based on the risk category and the impact.

8. The method of claim 7, wherein obtaining the patient information comprises access the patient information at least one remote health care system.

9. The method of claim 7, wherein the risk score is based on insurance claims data.

10. The method of claim 7, wherein the risk score is agnostic to healthcare costs.

11. The method of claim 7, wherein the patient information includes a plurality of risk scores.

12. The method of claim 7, wherein the impact is identified further based on a severity of the diagnosis.

13. One or more non-transitory computer readable media encoded with instructions which, when executed by one or more processors of a segmentation system, cause the segmentation system to:
obtain patient information for a plurality of patients, the patient information including at least a risk score;
cluster the plurality of patients based on the patient information using a categorization model, wherein the categorization model is trained using unsupervised learning; and
assigning a risk category to each patient based on the clustering.

14. The one or more non-transitory computer readable media of claim 13, wherein the instructions further cause the one or more processors to access the patient information at least one remote health care system.

15. The one or more non-transitory computer readable media of claim 13, wherein the risk score is agnostic to healthcare costs.

16. The one or more non-transitory computer readable media of claim 15, wherein the patient information includes a plurality of risk scores.

17. The one or more non-transitory computer readable media of claim 16, wherein the instructions further cause the one or more processors to calculate at least one risk score of the plurality of risk scores based on the patient information.

18. The one or more non-transitory computer readable media of claim 13, wherein the patient information includes a diagnosis, wherein the instructions further cause the one or more processors to identify, for a patient of the plurality of patients, an impact of at least one intervention based on the diagnosis and using an impact model.

19. The one or more non-transitory computer readable media of claim 13, wherein the instructions further cause the one or more processors to prioritize the patient based on the risk category and the impact.

20. A computer implemented method comprising:
obtaining patient information for a plurality of patients, the patient information including at least a diagnosis;
determining, using an impact model and based on the diagnosis, an impact of at least one intervention based on the diagnosis; and
prioritizing in a health services database at least a patient of the plurality of patients based on the impact of the at least one intervention.

21. A computer implemented method comprising:
receiving a request for a patient profile;
determining a risk category for the patient based at least on patient information associated with the patient, the patient information including at least a risk score, wherein the risk category is determined using an unsupervised machine learning model; and
presenting, via a user interface, the patient profile, wherein the patient profile includes the risk category for the patient.
